(19) United States
(12) Patent Application Publication
(10) Pub. No.: US 2004/0170686 A1
(43) Pub. Date: Sep. 2, 2004

(54) Suspension vehicle for coated drug particles

(76) Inventors: Jennifer K. Fredrickson, Kalamazoo, MI (US); Joseph P. Reo, Kalamazoo, MI (US)

Correspondence Address: Karen B. King, Warner-Lambert Company LLC, 2800 Plymouth Road, Ann Arbor, MI, 48105, US

(21) Appl. No.: 10/763,299

(22) Filed: Jan. 22, 2004

Related U.S. Application Data
(60) Provisional application No. 60/444,311, filed on Jan. 31, 2003.

(51) Int. Cl. A61K 9/26

(52) U.S. Cl. 424/469; 514/376

(57) ABSTRACT

The present invention is directed to a suspension vehicle for coated drug particles. The coated drug particles are suitable for use in taste masking. The suspension vehicle disclosed enables the maintenance of coated drug particles with an average particle size of about 50 μm to about 600 μm in suspension for an extended period of time, without sedimentation or floating, even after shaking in the presence of air. The suspension vehicle of the present invention makes it possible to produce multidose oral suspensions of coated drugs, from which consistent dosages can be dispensed over time. The dry formulations, methods, and suspension vehicles of the present invention are particularly well suited for the delivery of objectionable tasting drugs, such as linezolid.

### CROSS-REFERENCE TO RELATED APPLICATIONS

This application claims the benefit of U.S. Provisional Application Serial No. 60/444,311, filed Jan. 31, 2003.

### FIELD OF THE INVENTION

The present invention relates to suspensions of coated drugs, such as microencapsulated drug particles, and to means of maintaining the coated drug particles in suspension in a viscous solution over a long period of time, after shaking in the presence of air, without a significant incidence of settling out or floatation. The present invention particularly relates to multi-dose suspensions of coated drug particles, such as multi-dose suspensions of coated linezolid particles.

### BACKGROUND

Drug suspension formulations have long been used to orally administer drugs to young children, to the elderly, and to other persons including disabled or incapacitated persons who have trouble swallowing tablets or capsules. Suspension formulations are generally designed to be used either only once, with the solid form of the drug being suspended in a liquid immediately before use, or multiple times after the solid form of the drug is suspended in the liquid. Multi-use prescription suspensions are generally sent to pharmacies as a dry formulation to be added to and suspended in water or in another aqueous solution by immediately prior to sale.

Suspensions of coated drugs, such as microencapsulated drugs, have been observed to sediment and/or rise to the top of a suspension, over time. See, p. 2 of WO 98/17250 (EURAND INTERNATIONAL S.P.A.). Floating, rather than sedimentation, appears to be a considerably more common problem in suspensions of coated drugs. Sedimentation and/or floatation result in formulations that are not uniform in drug concentration. Use of such non-uniform formulations can result in overdosing or underdosing.

It is well known, among those who prepare pharmaceutical suspensions comprising a solid dispersed in a liquid medium, that the uniformity of the dispersed solid throughout the medium can be approximated using Stoke's Law, shown below, an approximation of Newton's second law wherein every particle is assumed to be a sphere:

v=d²(ρₚ−ρ₁)/18η, where:

v=settling velocity=ds/dt=distance traveled (up or down) over time

d=Stokes diameter (equivalent spherical diameter)

ρₛ=density of solid

ρ₁=density of liquid

g=acceleration due to gravity

η=viscosity of the dispersion medium (liquid)

When the settling velocity (v) is positive, one would expect suspended particles move in a downward direction (sink or sediment). When the settling velocity is negative, one would expect the particles move in an upward direction (float). For a good discussion of Stokes law and its use in predicting floating and sedimentation in suspensions, see Dell, Sheila M. et al., “Avicel® RC/CL Microcrystalline Cellulose and Carboxymethylcellulose Sodium, NF, BP”, Section 14, pub. by FMC BioPolymer (2001) pp. 1-27, at pp. 2-5. See, also, Robinson, J. V., “Rise of Air Bubbles in Lubricating Oils,” Technical Note 2033, National Advisory Committee for Aeronautics, Washington, (February 1950), pp. 1-24, at pp 1-6.

In order to formulate a pharmaceutical suspension, the density of the solid, density of the liquid, the particle size of the solid and the viscosity of the liquid must all be manipulated in order to minimize particle movement in the dispersion medium i.e. particle movement due to gravitational force, buoyancy force and drag force. The goal is to make the difference, (ρₛ−ρ₁) approach zero, increase the viscosity of the liquid but not so much as to make the suspension unusable (i.e. too thick to pour) and the particle size of the solid as small as possible, but not too small as to cause aggregation due to electrostatic attraction. When pharmaceutical drug particles are coated, interactions between the suspension medium and the coating, or even between the coating and air in the medium can make it considerably more complicated to produce a homogeneous suspension of such drug particles.

Multi-dose, ready-to-use pharmaceutical suspensions of drug particles are generally provided as a dry formulation in a bottle, to which either a pharmacist or consumer adds water, and is instructed to shake vigorously to dissolve the dissolvable solids and to disperse the dispersible solids. When the suspension is shaken in the presence of air, initially or at any other time prior to using, air is inherently introduced into the dispersion. The air becomes dispersed in the resulting dispersion medium, along with the solid particles. What happens to the dispersed air depends on certain factors. For example, if the viscosity is such that the air bubbles collide with each other, they will form larger air bubbles (called coalescence). The larger air bubbles have even higher buoyancy and will rapidly float to the top of the dispersion and, if liquid film around the air bubble is relatively weak, the film will rupture, and the air bubble will collapse, releasing the air into the atmosphere above the dispersion. In other words, the air escapes fairly rapidly. However, if the viscosity of the liquid is higher, reducing the bubble velocity, there is a greater chance for air bubbles to collide with solid particles. If the air spreads on the surface of the solid particles (i.e. the interfacial tension between the solid and air favors spreading/adsorption) and if the surface of the solid particle is at least partially hydrophobic will tend to become attached to the air bubbles, and the air and solid will form a solid-air aggregate particle, referred to as an “aerofloc.” Surface active agents, like surfactants, which are usually present in a suspension formulation, increase the likelihood of aerofloc formation. For a good general discussion of the interaction between air bubbles and a different type of solid particle, minerals, in suspensions, see Perry's Chemical Engineer's Handbook, 7^{th} ed., Don Green, editor (pub. by McGraw-Hill, 1997), pp 56-65.

Due to the very low mass of the air portion of the solid-air aggregate particles, the density of the aggregate is considerably less than that of the solid alone. The lower density of the aggregate usually causes the aggregate to move upward, and in many instances, causes the aggregate to float, resulting in solid separation and non-uniform suspension. Non-uniform suspension will result in non-homogeneous dosing from the container and an unacceptable product.

Various approaches have been taken to improve the effectiveness of the suspension of coated drugs, particularly, in terms of homogeneity. However, none cited below appear to have taken into account the role played by formation of solid-air aggregates on the non-uniformity of pharmaceutical suspensions, described immediately above.

U.S. Pat. No. 5,306,506 (Zema et al.) describes a solid pharmaceutical composition of a micro-encapsulated drug designed to be added to water to produce a monodose suspension. The solid composition includes the microencapsulated drug or a “drug which is substantially water-insoluble,” a “thickening or suspending agent,” a “pharmaceutically accepted acid,” and a particular weight ratio of “a pharmaceutically acceptable carbonate or bicarbonate . . . sufficient to obtain rapid hydration of the thickening or suspending agent when mixed with water.” (language of claim 1). The acidic substance and base are included in order to avoid effervescence, as “the formation of bubbles of carbon dioxide tends to carry afloat the granules coated with the thickening agent . . . ” (Id., col. 3, lines 26-31). The '506 patent states that when water is added to a monodose dry formulation composition of that invention, the thickening agent confers sufficient viscosity to the resulting medium “to maintain the microcapsules in a homogeneous suspension in order to avoid the formation of lumps and especially separation of the microcapsules (floating and sedimentation).” ('506 patent, col. 4, lines 24-35.) Many different thickening agents are listed as being of possible use in the composition disclosed therein, including xanthan gum, and crystalline cellulose alone, or in combination with other hydrocolloids (e.g., Avicel® RC-591 of FMC Corp.). ('506 patent, col. 5, lines 20-26.)

Although the '506 patent appears to address the problem of floating and sedimentation in single-use suspensions, the only form of solid pharmaceutical compositions disclosed therein are “monodose saches.” Such monodose saches are designed for rapid suspension and immediate, single-time use, not for maintenance of suspend ability over time, a desired property for multi-use suspensions. For descriptions of other monodose suspensions of coated drugs with similar thickening agents and other methods of producing the same in ways that reportedly control floatation and/or sedimentation, see U.S. Pat. No. 5,008,117 (Calanchi et al.), U.S. Pat. No. 6,261,602 (Calanchi et al.), and International Publication Number WO 01/52848 (EURAND AMERICA, INC.).

Use of thickening agents or mixtures of thickening agents to obtain multi-dose homogeneous suspensions of uncoated drugs is known. See, for example, U.S. Pat. No. 4,788,220 (Mody et al.), U.S. Pat. No. 5,272,137 (Blase et al.), U.S. Pat. No. 5,409,907, and International Publication Number WO 99/63937 (ADVANCED MEDICINE, INC.). However, for reasons given herein above, one would not expect that materials and methods which work to produce multi-dose homogeneous suspensions of uncoated drugs would work to produce multi-dose homogeneous suspensions of one or more drugs at least partially coated with a hydrophobic polymer film coating.

Linezolid, an oxazolidinone antibiotic drug, has an offensive taste when suspended in an aqueous solution without any taste-masking components. Microencapsulation of the drug for incorporation into chewable tablets has been disclosed in International Publication Number WO 01/52848 (EURAND INTERNATIONAL S.P.A.).

What is needed is a means for obtaining a homogeneous suspension of coated linezolid, in the presence of air, where the bad taste of the drug is controlled by the microcapsule alone or in combination with the surrounding solution.

What is also needed is a means for producing a multi-dose suspension of coated drug particles, in the presence of air, in general, a suspension where the coated drug particles neither sediment nor float out of solution over time even when the suspension solution is viscous, after being shaken in the presence of air.

As is illustrated below, the present invention meets both of these needs.

### BRIEF SUMMARY OF THE INVENTION

The present invention relates to a dry formulation for preparing substantially homogeneous suspensions of drug particles coated, at least in part, with a hydrophobic polymer film, and to suspensions of such drug particles. The suspensions of the present invention maintain homogeneity in the presence of air, under conditions under which one would not expect homogeneity to be established or maintained, based upon Stokes Law and other general principals of suspension dynamics, summarized above. Unexpectedly, a substantially homogeneous suspension of the coated drug particles is produced when suspensions of the present invention or when suspensions of the dry formulation of the present invention have a viscosity higher than what one would select by applying Stokes law. Specifically, it is believed that the suspensions of the present invention prevent the solid-air particles from appreciably forming, to the extent substantial homogeneity is maintained throughout suspensions of the present invention, even in the presence of air.

Ordinarily, one would raise the viscosity of a suspension to reduce particle movement in a downward direction. However, as described above, for multi-dose suspensions, which need to be shaken before a dose is dispensed, a process that incorporates air into the suspension, raising viscosity reduces the migration rate and air-air collision rate of bubbles, and air becomes entrapped in the suspension. The entrapped air can associate with hydrophobic-coated particles, such as the drug particles of the present invention, and reduce the effective density of the solid-air aggregate. The dry formulations of the present invention form suspensions with a homogeneous dispersion of air bubbles and solid particles that do not interact with each other, even after vigorous shaking. Thus, if a solid-air aggregate forms, the viscosity of the resulting suspension is sufficiently high to slow movement of the aggregates within the suspension to prevent non-uniformity.

The coated drug particles included in the formulations and suspensions of the present invention each comprise a core, comprising a drug and a polymer film, coating at least a portion of the core.

One embodiment of the invention relates to a dry formulation comprising (a) at least two doses of the coated drug particles, having an average particle size of about 50 μm to about 600 μm; and (b) a viscosity enhancing substance in an amount effective to maintain the at least two doses of coated drug in a substantially homogeneous suspension for at least 24 hours at about 20° C. to about 30° C., after combination with about 2 ml to about 60 ml of an aqueous liquid per dose of the coated drug and mixing in the presence of air.

Another embodiment relates to a dry formulation comprising at least two doses of the coated drug particles, having an average particle size of about 50 μm to about 600 μm, xanthan gum, microcrystalline cellulose and sodium carboxymethylcellulose, wherein the weight ratio of the xanthan gum to the microcrystalline cellulose and the carboxymethylcellulose is about 1:2 to about 1:0.3, wherein a suspension with a viscosity of at least about 1500 cps is formed after combination of the dry formulation with about 2 ml to about 60 ml of an aqueous liquid per dose of the coated drug particles.

Another embodiment relates to a method of producing a multi-dose suspension of the coated drug particles, having an average particle size of about 50 μm to about 600 μm, comprising the steps of: (a) providing a dry formulation comprising at least two doses of coated drug particles, xanthan gum, microcrystalline cellulose and sodium carboxymethylcellulose, wherein the weight ratio of xanthan gum to microcrystalline cellulose and carboxymethylcellulose is about 1:2 to about 1:0.3; and (b) combining the dry formulation with an aqueous solution for a viscosity of at least about 1500 cps, and agitating the same until a suspension is formed.

In another embodiment, the method of treating or preventing a gram-positive bacterial infection in a subject comprises orally administering to a subject at least two doses of a multi-dose suspension of coated linezolid particles, produced as described above.

The dry formulations of the present invention produce very stable suspensions of coated drug, suspensions where the coated drug particles neither float nor sediment over a long period of time, even after shaking in the presence of air, making production of multi-dose formulations of such coated drug particles possible. These and other properties of the dry formulations of the invention, and methods of making and using suspensions produced therefrom are further illustrated herein below.

### BRIEF DESCRIPTION OF THE DRAWINGS

FIG. 1 is a plot of dose dependency over settling time since reconstitution, from each of two bottles of a formulation (Formulation A) of suspended microencapsulated linezolid particles prepared and tested as described in Example 2.

FIG. 2 is a plot of weight of dose dependency on time since constitution of three different samples of a formulation (Formulation C) of microencapsulated linezolid particles prepared, as described in Example 5, with a ratio of xanthan gum to microcrystalline cellulose and carboxymethylcellulose of about 1:0.8, and tested as described in Example 6.

FIG. 3 is a plot of dose dependency over settling time since reconstitution, from three sample suspensions of the same formulation (Formulation C) of microencapsulated linezolid particles prepared as described in Example 5, and tested as described in Example 6.

### DETAILED DESCRIPTION OF THE INVENTION

The term “microencapsulated”, as used herein, indicates a micron sized core comprising substances in the form of particles, powders, crystals, granules, pellets, and liquid drops, coated with a continuous polymeric film.

As used herein, the term “microencapsulated drug particle” refers to a core comprising a drug or combination of drugs alone or in combination with excipients, wherein the core has been microencapsulated.

The term “microencapsulation”, as used herein, refers to a process consisting of coating a micron sized core with a continuous polymeric film.

The term “oral administration” herein includes any form of delivery of a therapeutic agent or a composition thereof to a subject wherein the agent or composition is swallowed by a subject, regardless of whether the composition is placed in the mouth prior to swallowing. Thus “oral administration” includes esophageal administration. Absorption of the agent can occur in any part or parts of the gastrointestinal tract including the mouth, esophagus, stomach, duodenum, ileum and colon.

The term “orally deliverable” herein means suitable for oral administration.

A “subject” herein to which a therapeutic agent or composition thereof can be administered includes a human patient of either sex and of any age, and also includes any nonhuman animal, particularly a domestic or companion animal, illustratively a cat, dog or horse.

The term “dose” herein means an amount of a drug or pharmaceutical formulation to be taken or applied all at one time or in fractional amounts within a given period. In the case of an oral suspension, a dose is an amount of the suspension to be taken orally at once, or in fractions one after another at a given time period.

The term “multidose” as used herein, refers to at least two doses of a drug or pharmaceutical formulation.

The term “multidose sachet” is a container which contains at least two doses of a drug and excipients in a dry formulation.

The term “present in solid particles” as applied to a drug herein encompasses compositions wherein the solid particles consist essentially of the drug and compositions wherein the solid particles comprise the drug in intimate mixture with one or more other ingredients. These other ingredients can include one or more therapeutic agents other than the drug and/or one or more pharmaceutically acceptable excipients.

The term “excipient” herein means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition.

As used herein, the term “stable suspension” refers to a suspension of particles wherein the particles remain in suspension, with no visible floating or sedimentation, for at least 24 hours with no mixing after an initial suspension step.

The term “viscosity enhancing substance”, as used herein, refers to substances which dissolve in water and which increase in density and viscosity, allowing solid particles to be suspended therein.

The term “substantially homogeneous suspension”, as used herein, refers to a suspension of solid material in a solution, such as a suspension of microencapsulated drug in a solution, wherein substantially uniform dosing is possible throughout the suspension.

The coated drug particles of the present invention can suitably comprise any drug or combination of drugs that are at least slightly soluble in water. The drug is preferably an antibiotic, more preferably an oxazolidinone antibacterial drug, even more preferably an oxazolidinone antibacterial drug compound of formula (I)

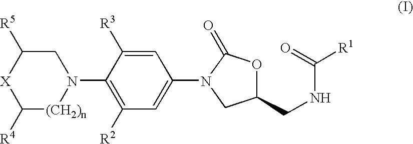

wherein:

R¹ is selected from (a) H, (b) C₁₋₈ alkyl optionally substituted with one or more of F, Cl, OH, C₁₋₈ alkoxy, C₁₋₈ acyloxy or benzoxy groups, and including C₃₋₆ cycloalkyl, (c) amino, (d) mono- and di(C₁₋₈ alkyl)amino and (e) C₁₋₈ alkoxy groups;

R² and R³ are independently selected from H, F and Cl groups;

R⁴ is H or CH₃;

R⁵ is selected from H, CH₃, CN, CO₂R¹ and (CH₂)ₘR⁶ groups, where R¹ is as defined above, R⁶ is selected from H, OH, OR¹, OCOR¹, NHCOR¹, amino, mono- and di(C₁₋₈ alkyl)amino groups and m is 1 or 2;

n is 0, 1 or 2; and

X is O, S, SO, SO₂, SNR⁷ or S(O)NR⁷ where R⁷ is selected from H, C₁₋₄ alkyl (optionally substituted with one or more F, Cl, OH, C₁₋₈ alkoxy, amino, C₁₋₈ mono- or di(C₁₋₈ alkyl)amino groups), and p-toluenesulfonyl groups;

or a pharmaceutically acceptable salt thereof.

A particularly preferred embodiment of the oxazolidinone antibacterial drug is a compound of formula (II), wherein R¹ is CH₃; R² and R³ are independently selected from H and F but at least one of R² and R³ is F; R⁴ and R⁵ are each H; n is 1; and X is O, S or SO₂. In another preferred embodiment, the oxazolidinone antibacterial drug is selected from the group consisting of: linezolid, eperezolid, N-((5S)-3-(3-fluoro-4-(4-(2-fluoroethyl)-3-oxopiperazin-1-yl)phenyl)-2-oxooxazolidin-5-ylmethyl)acetamide, (S)-N-[[3-[5-(3-pyridyl)thiophen-2-yl]-2-oxo-5-oxazolidinyl]methyl]acetamide, (S)-N-[[3-[5-(4-pyridyl)pyrid-2-yl]-2-oxo-5-oxazolidinyl]methyl]acetamide hydrochloride and N-[[(5S)-3-[4-(1,1-dioxido-4-thiomorpholinyl)-3,5-difluorophenyl]-2-oxo-5-oxazolidinyl]methyl]acetamide.

Linezolid is a particularly preferred oxazolidinone antibacterial drug incorporated into the coated drug particles of the present invention. Linezolid is known to exhibit strong antibacterial activity against gram-positive organisms including those of the following genera: Staphylococcus (e.g., Staphylococcus aureus, Staphylococcus epidermidis), Streptococcus (e.g., Streptococcus viridans, Streptococcus pneumoniae), Enterococcus, Bacillus, Corynebacterium, Chlamydia and Neisseria. Many such gram-positive organisms have developed significant levels of resistance to other antibiotics.

The present invention is illustrated herein with particular reference to linezolid. However, it will be understood that it is contemplated that other drugs, including other antibiotics or other oxazolidinone antibacterial compounds, such as those of formula (I), above, could be substituted in whole or in part for linezolid. In some cases, it will be necessary to make appropriate adjustment in concentration and dosage ranges to account for properties of the particular type of drug or combination of drugs included in the coated drug particles used in the present invention, as described herein.

The coating of each coated drug particle used in the dry formulations, suspensions, and methods of the present invention preferably reduces the availability of the drug compared to a suspension of uncoated drug, while not adversely impacting the bioavailability of the drug. The polymer coating preferably coats at least 70% of the drug in the core of each coated drug particle, more preferably at least 80% of the drug in the core, even more preferably at least 90% of the drug in the core. In a preferred embodiment of the present invention, hereinafter referred to as “microencapsulated drug particles,” at least one polymer film fully encapsulates each drug particle.

The coated drug particles of the present invention can suitably be produced by any one of a number of known means of coating of core particles, including means described in Reo & Fredrickson, “Tastemasking Science and Technology Applied to Compacted Oral Solid Dosage Forms—Part 2, Amer Pharm Rev (Fall 2002), pp. 2-13, incorporated by reference herein. Suitable means of microencapsulation for use in producing the suspensions and in practicing the methods of the present invention are disclosed in the above-cited article by Reo & Fredrickson, and in U.S. Pat. No. 3,196,827 (Wurster et al.), U.S. Pat. No. 3,253,944 (Wurster et al.), U.S. Pat. No. 3,415,758 (Powell et al.), U.S. Pat. No. 3,155,590 (Miller et al.), U.S. Pat. No. 3,341,416 (Anderson et al.), U.S. Pat. No. 5,008,117 (Calanchi et al.), U.S. Pat. No. 6,261,602 B1 (Calanchi et al.), and U.S. Pat. No. 6,139,865 (Friend et al.), all of which are incorporated herein by reference. The particular coating method selected depends upon the physical and chemical characteristics of the drug to be microencapsulated. For example, when the drug is in the form of a liquid, the polymer film and method used to coat the drug in the film is preferably one that is effective in containing the liquid in both a dry formulation and in a suspension medium. In contrast, drugs in the form of particles or crystals can be coated with any one of a wide variety of different pharmaceutically acceptable polymer films. The drug in the formulations of the present invention is preferably in the form of drug particles or drug crystals, more preferably in the form of drug particles.

Hydrophobic polymers suitable for use as the polymer film of the coated particles used in the present invention include, but are not limited to, vinyl acetate, vinyl chloride, vinyl carbonate, methacrylic acid, polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate. Ethyl cellulose, cellulose acetate phthalate methacrylic acid, and polymethacrylic acid copolymer are preferred, with methacrylic acid, and polymethacrylic acid copolymers being particularly preferred.

Some hydrophobic polymers, such as ethylcellulose can be processed in such a way that they form a microparticulate coacervate with a drug, another form of coated drug particles suitable for use in the formulations and suspensions of the present invention. Some such coacervates will completely encapsulate a drug. However, to ensure complete encapsulation, it is possible to add a coating of a second polymer to the coacervate.

The pharmaceutically acceptable polymer film suitably comprises at least two layers, such as an inner layer with the capacity to delay drug release, such as ethylcellulose or a coacervate of drug and ethylcellulose, and an outer hydrophobic polymer layer, such as polymethacrylate, that dissolves on a pH dependent basis. The method used to produce the microencapsulated drug included in the dry formulation or suspension of the present invention depends upon the physical and chemical characteristics of the drug and of the polymer used to produce the polymer film. For suitable methods for use in producing the microencapsulated drug particles included in the formulations and suspensions of the present invention, see Reo & Fredrickson, supra, and WO 99/52510 (EURAND INTERNATIONAL SPA), all of which are incorporated by reference herein. Reo and Fredrickson (supra), specifically, review and evaluate numerous polymer film and substrate particle, crystalline, and matrix configurations described in the literature. Any one of the configurations utilizing hydrophobic polymer films disclosed therein would be suitable for use in the methods and suspensions of the present invention.

Regardless of whether the coated particles include one or more coating layers of hydrophobic polymer, at least one layer of polymer film coating preferably includes a plasticizer deposited thereon or incorporated therein. When the coated drug particles include at least two coating layers of polymer film, the outer layer is preferably plasticized pharmaceutical grade shellac, Colorcon Opadry, or a plasticized hydroxypropylmethylcellulose formulation.

The hydrophobic polymer coating of a coated drug particle, particularly when the coated drug particle is microencapsulated, can delay release of a drug in suspension until after administration to a subject. When administration is oral and the drug is one with an offensive taste, microencapsulation can mask the offensive taste by delaying release until after the drug formulation has passed through the mouth of a subject. Even partial coating of a drug with a hydrophobic polymer coating, as described above, can delay release of a drug, both in suspension and after administration to a subject, decreasing any offensive drug taste. Such factors are particularly important when the subject is one likely to reject offensive tasting drugs. The drug or combination of drugs in the core of the coated drug particles used in the formulations, suspensions, and methods of the present invention is preferably drug or combination of drugs with an offensive taste when taken orally.

For reasons set forth immediately above, the present invention is particularly well suited for use in the oral administration of offensive tasting drugs, such as offensive tasting antibiotics, including offensive tasting oxazolidinone antibiotics, more specifically including linezolid. Taste-masking of linezolid by microencapsulation has been described in International Publication Number WO 015248 A2 (EURAND AMERICA, INC.), incorporated by reference herein.

It is preferable to minimize the number of excipients in the core, in order to minimize any possible interference with taste masking of the drug. In one embodiment, the core of each coated drug particle consists solely of the drug.

In an alternative embodiment, the core of the coated drug particles further comprise the drug admixed with at least one core excipient selected from the group consisting of pharmaceutically acceptable diluent, binding agent, adhesive, wetting agent, lubricant, plasticizer, and anti-adherent agent. Through selection and combination of core excipients, compositions can be provided exhibiting improved performance with respect to, among other properties, efficacy, bioavailability, clearance time, stability, compatibility of drug and excipients, safety, dissolution profile, and/or other pharmacokinetic, chemical and/or physical properties. Preferably, the amount and number of excipients in the core is minimized in order to avoid adversely affecting the taste or mouth feel of the drug, upon oral administration.

When at least one core excipient is a diluent, the diluent is suitably lactose, including anhydrous lactose and lactose monohydrate; a starch, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, and amorphous cellulose (e.g., Rexcel™) and powdered cellulose; calcium carbonate; glycine; bentonite; polyvinylpyrrolidone; and combinations of any of the above.

Microcrystalline cellulose is a preferred diluent. This diluent is chemically compatible with linezolid. Inclusion of microcrystalline cellulose in the core of coated drug particles can improve hardness and/or disintegration time of the particles. Microcrystalline cellulose typically provides compositions having suitable release rates of drugs admixed therewith, stability, flowability, and/or drying properties at a relatively low diluent cost.

The core of coated drug particles optionally comprise at least one pharmaceutically acceptable binding agent or adhesive as a core excipient. Such binding agents and adhesives preferably impart sufficient cohesion to the core while allowing the particles to disintegrate and the drug to be absorbed after the drug particles pass through the mouth and into the remainder of the gastrointestinal tract of a subject, after ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, microcrystalline cellulose, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™).

The coated drug particles optionally comprise one or more pharmaceutically acceptable disintegrants as excipients. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

The coated drug particles optionally comprise at least one pharmaceutically acceptable wetting agent as a core excipient. Non-limiting examples of plasticizers suitable for use as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof.

The core of the coated particles optionally comprises at least one pharmaceutically acceptable lubricant, as a core excipient. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; DL-leucine; PEG (e.g., Carbowax™ 4000 and Carbowax™ 6000); sodium oleate; sodium lauryl sulfate; and magnesium lauryl sulfate. The lubricant is preferably an anti-adherent. Suitable anti-adherents include talc, cornstarch, DL-leucine, sodium lauryl sulfate, colloidal silica, and metallic stearates. Talc is a preferred anti-adherent or glidant used, for example, to reduce formulation sticking to equipment surfaces and also to reduce static in the blend.

The dry formulation of the present invention is preferably provided in a multidose sachet, comprising at least two doses of the coated drug. The multidose sachet of the dry formulation can be used to produce a multi-dose suspension of the coated drug by combining an aqueous liquid therewith.

Dosage is determined by a combination of a number of factors, such as age, weight, size, and general physical condition of a subject, as well as on the particular drug present in the formulation, the potency of the coated drug and on other medication being administered to the subject. When the drug is linezolid, and the subject is a human pediatric subject, the dosage is preferably about 5 to about 15 mg of linezolid per kilogram (“Kg”) body weight, more preferably about 10 mg linezolid per Kg of body weight.

The multi-dose dry formulation of the present invention is preferably suspended in a volume of aqueous liquid that enables one to orally administer each dose of the coated drug to a subject in a reasonable volume. The volume is preferably about 2 ml to about 60 ml per dose, more preferably about 5 ml to about 50 ml per dose, even more preferably about 5 ml to about 30 ml per dose. When the subject is a human infant or small human child, the volume is preferably limited to about 2 ml to about 20 ml, more preferably to about 5 ml to about 15 ml.

The aqueous liquid can be any aqueous liquid suitable for use in suspending the coated drug in the suspension vehicle of the present invention. Suitable aqueous liquids include aqueous buffer solutions, alcohol solutions, and water. The aqueous liquid is preferably water. Although any type of water is suitable for use in the suspensions of the present invention, the water is preferably purified water, more preferably spring water, more preferably deionized water, even more preferably deionized distilled water.

The coated drug is present in the formulations of the present invention at a concentration that enables one to orally administer at least one dose of the coated drug per day to a subject. When the drug is linezolid, the daily amount of coated drug administered to a human adult subject is preferably about 100 mg to about 1000 mg, more preferably about 200 mg to about 750 mg, even more preferably about 600 mg of linezolid. When the drug is linezolid, the daily amount of coated drug administered to a human pediatric subject is preferably about 40 mg to about 600 mg, more preferably about 50 mg to about 300 mg. For other drugs, a daily dose that is therapeutically equivalent to the above dose ranges for linezolid is preferably administered.

The suspension vehicle of the present invention can be used to suspend a broader particle size range of coated drug particles than known suspension vehicles. However, the smaller the particles and the narrower the size range, the more likely all the particles will remain suspended in any given suspension vehicle without floating or sedimentation. The coated drug particles included in the dry formulations and suspensions of the present invention preferably has a particle size range that enables the coated drug particles to be readily suspended in a suspension formulation of the present invention and remain substantially uniformly suspended therein for at least 24 hours at about 20° C. to about 30° C. The coated drug particles preferably have an average particle size of about 50 microns (hereinafter, “μm”) to about 600 μm, more preferably an average particle size of about 75 μm to about 400 μm, more preferably an average particle size of about 100 μm to about 250 μm, even more preferably an average particle size of about 100 μm to about 180 μm.

The suspension formulation preferably comprises a viscosity enhancing substance in an amount effective to maintain the coated drug particles in suspension for at least 24 hours at 20° C. to about 30° C., more preferably at room temperature (i.e., at about 25° C.) after combination with an amount of aqueous liquid selected as described above. When present at an appropriate concentration for the specific viscosity enhancing substance, the substance acts as a suspension enhancer. The viscosity of the resulting suspension is preferably sufficiently low that the suspension has good flow characteristics, in order to facilitate oral administration. The viscosity of the suspension of coated drug particles, after addition of an aqueous liquid to the dry formulation, is preferably at least about 1,500 cps, more preferably about 1,500 cps to about 4,500 cps, even more preferably about 2,000 cps to about 4,100 cps, even more preferably about 2,400 to about 3,800 cps.

The viscosity enhancing substance is preferably selected from the group consisting of an alginate, carageenin, agar-agar, tragacanth gum, xanthan gum, guar gum, caroba gum, karaya gum, modified corn starch, carboxymethyl cellulose, and crystalline cellulose alone or in combination with other hydrocolloids. The viscosity enhancing substance preferably comprises xanthan gum or a mixture of xanthan gum and at least one other viscosity enhancing substance, such as microcrystalline cellulose and carboxymethylcellulose. The viscosity enhancing substance is most preferably a mixture of xanthan gum, microcrystalline cellulose, and carboxymethylcellulose. When the viscosity enhancing substance is the mixture cited immediately above, a weight ratio of xanthan gum to microcrystalline cellulose and carboxymethylcellulose is selected that is effective in maintaining the coated drug particles in suspension. That weight ratio depends, furthermore upon the average particle size of the drug particles. Specifically, when the coated drug particle size is 30 microns to about 600 microns, the weight ratio of xanthan gum to microcrystalline cellulose and carboxymethylcellulose is preferably about 1:4 to about 1:0.2, more preferably about 1:2 to about 1:0.3, most preferably about 1:0.8.

The formulations of the present invention preferably further comprise at least one taste-masking substance. The at least one taste-masking substance is preferably a sugar, even more preferably a sugar selected from the group consisting of lactose, mannitol, sucrose, glucose, or a mixture of the above. The sugar is most preferably sucrose. When the sugar is sucrose, it is preferably about 35% to about 60% by weight, more preferably about 40% to about 55% by weight, even more preferably about 45% to about 50% by weight, of the dry formulation of the present invention. This same dry formulation is preferably used to make the suspension of the present invention.

At least one taste-masking substance is suitably an artificial sweetener, a flavoring agent, or a combination of a sugar and at least one artificial sweetener or flavoring agent.

Any flavoring agent is suitable for inclusion in the formulations of the present invention, when the drug is suitably taste-masked in the absence of the flavoring agent. Flavoring agents are also suitable for use, that mask detectable objectionable tastes or other unpleasant flavors found to be present in some suspensions of dry formulations of the present invention, in the absence of such flavoring agents.

The coated drug particles are preferably suspended within 30 minutes of when the aqueous liquid is combined with the dry formulation of the present invention, more preferably within 20 minutes, more preferably within 5 minutes, even more preferably within 3 minutes of being combined therewith.

In another embodiment, the present invention relates to a method of producing a multi-dose suspension of coated drug particles described above from a dry formulation of the present invention. A dry formulation comprising the drug, xanthan gum, microcrystalline cellulose, and sodium carboxymethylcellulose, with a weight ratio of xanthan gum to microcrystalline cellulose and carboxymethylcellulose of about 1:2 to about 1:0.2 is provided, and combined with an aqueous solution and agitated until a homogeneous suspension is formed. The drug is preferably an oxazolidinone, more preferably linezolid. When the drug is linezolid, the weight ratio of xanthan gum to microcrystalline cellulose and carboxymethylcellulose is more preferably about 1:2 to about 1:0.3, more preferably about 1:2 to about 1:0.7, even more preferably about 1:0.8. Other preferred features and optional additional components of the dry formulation described above are also suitable for use in the method of producing a multi-dose suspension of the present invention.

In another embodiment, the present invention relates to a method of using a suspension of a dry formulation of the present invention, wherein the coated drug particles are coated oxazolidinone antibiotic drug particles, to treat or prevent an a gram-positive infection in a subject. The oxazolidinone antibiotic drug is preferably linezolid. The method comprises orally administering at least two doses of a suspension of a dry formulation of the present invention to a subject who either has a gram-positive infection or who is at risk of contracting a gram-positive infection. Preventative use is appropriate, for example, prior to or after invasive surgery, or after a subject has contracted an open wound that has not yet become infected. Preferred features and optional suitable components of the suspension suitable for use in the method of the present invention are described herein above.

The present invention is further illustrated by the following examples. These examples are intended to be illustrative of the invention and should not be used to limit or restrict its scope.

### EXAMPLES

### Example 1

### Microencapsulation of Linezolid

Microencapsulated linezolid particles were supplied by Eurand America Corp. Microencapsulation methods of production are disclosed in WO 01/52848 (EURAND AMERICA, INC.), incorporated by reference herein. The microencapsulated linezolid particles used in the Examples, below, were formed by producing a coacervate of linezolid and ethylcellulose, followed by a first coating with ethylcellulose, and a second coating with polymethyacrylate. See, for example, pp. 3-4 of WO 01/52848, Id.

### Example 2

### Suspension of Microencapsulated Linezolid Particles

Microencapsulated linezolid particles produced as described in Example 1 were added to a placebo blend (i.e., a dry suspension formulation with no drug particles) in each of two different bottles. The placebo blend formulation used was based upon a pediatric suspension formulation of linezolid currently sold by Pharmacia Corporation, under the brand name ZYVOX®. The same amount of water was added to each bottle of the final blend, and the particles were suspended therein by shaking the resulting mixture for three minutes on a platform shaker. The composition of the dry formulation prior to suspension in water is given in Table 1, below.

TABLE 1

| mg/5 ml | % by | | Quantity |
|---|---|---|---|
| Dose | Weight | Component | in grams |
| 218 | 10.9 | Microencapsulated | 98.0 |
| | | Linezolid Particles | |
| 15.0 | 0.8 | Xanthan Gum | 11.2 |
| 50.0 | 2.5 | Microcrystalline Cellulose | 18.0 |
| | | and Sodium | |
| | | Carboxymethylcellulose | |
| | | (Avicel ® RC-591) | |
| 935 | 46.8 | Sucrose | 420.8 |
| 500 | 25.0 | Mannitol | 225.0 |
| 15 | 0.8 | Sodium Citrate | 6.75 |
| 9.1 | 0.5 | Citric Acid | 4.10 |
| 10 | 0.5 | Sodium Benzoate | 4.50 |
| 13.5 | 0.7 | Sodium Chloride | 6.08 |
| 90 | 4.5 | Artificial Sweeteners | 40.5 |
| 94.5 | 4.8 | Natural & Artificial Flavors | 42.5 |
| 2000 | | Total Powder Weight/5 ml dose | 900.0 |
| | | Total Weight (g) | |
| | | Number of Doses | 450.0 |

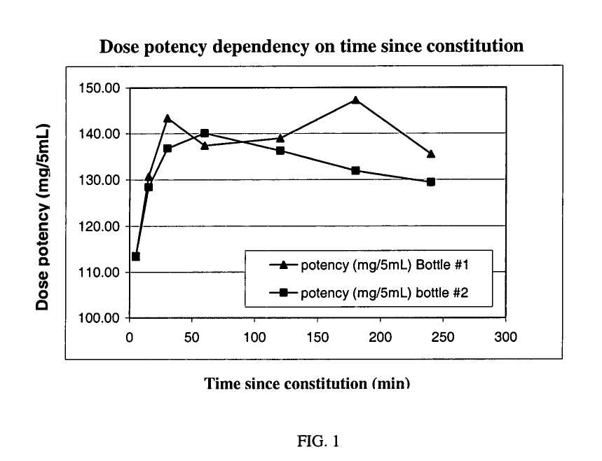

5 ml samples of the resulting suspension were poured out of each bottle at various time points. The suspensions were not shaken after the initial shaking step described above. Potency of linezolid in each sample was tested. The results obtained from this drug potency study are given in Table 2, below, and illustrated in graphic form in FIG. 1, below.

TABLE 2

| | Bottle 1 | Bottle 2 |
|---|---|---|
| Sit Time | Potency | Potency |
| (minutes) | (mg/5 ml) | (mg/5 ml) |
| 5 | 113 | 113 |
| 15 | 131 | 128 |
| 30 | 143 | 137 |
| 60 | 137 | 140 |
| 120 | 139 | 136 |
| 180 | 147 | 132 |
| 240 | 135 | 129 |

As on can see from Table 2 and from FIG. 1, the potency of drug in samples dispensed after the first five minutes was considerably higher than at the five minute mark. The drug potency continued to increase in each bottle tested after the first 15 minutes of sitting, after the initial shaking step. Both increases appear to be due to the fact that coated linezolid drug particles were observed rising to the top of each bottle, within the first five minutes of sitting, after suspension.

### EXAMPLE 3

### Effect of Sieve Cut on Homogeneity of Suspensions

The following study was conducted in order to determine whether the floating problem observed, above, could be alleviated by controlling the range of particle sizes of microencapsulated linezolid particles suspended in the formula. A dry formulation of placebo blend was produced, as described in Example 2, above, and placed in separate containers, to which were added three different sieve cuts of microencapsulated linezolid particles produced as described in Example 1 were added. US Standard sieves (30/60 mesh, 60/80 mesh, and 80/100 mesh) were used.

9.17 g samples of the dry formulation blends containing each sieve cut of microencapsulated linezolid particles, prepared, as described immediately above, were placed in separate 50 ml graduated cylinders. 9.7 ml of water was added to each graduated cylinder and shaken vigorously until each blend was reconstituted. An additional 9.7 ml water was added to each cylinder, and the cylinder shaken for an additional two to three minutes. Physical observations of each suspension were made and recorded immediately, and at various time points thereafter. Each suspension appeared to be homogeneous upon reconstitution. Observations made at each time point thereafter are summarized in Table 3, below:

TABLE 3

| Sample | Time Point | | Observation |
|---|---|---|---|
| 60 mesh Microencap- | 20 | min. | Suspension still homogeneous. |
| sulated Linezolid | | | |
| (U.S. standard | | | |
| 30/60 mesh cut) | | | |
| | 50 | min. | Suspension still homogenous, |
| | | | except for clearing at first |
| | | | 4 ml. at bottom of cylinder. |
| | 24 | hrs. | Significant amount of floating |
| | | | particles. Definite agglomeration |
| | | | of particles observed. |
| | | | First 5 ml. at bottom of the |
| | | | cylinder clear. |
| | 48 | hrs. | Agglomerated particles and |
| | | | floating particles observed. |
| 80 mesh Microencap- | 20 | min. | Suspension still homogenous. |
| sulated Linezolid | | | |
| (U.S. standard | | | |
| 60/80 mesh cut) | | | |
| | 20 | min. | Suspension still homogenous, |
| | | | except for some clearing at |
| | | | the bottom of the cylinder. |
| | 24 | hrs. | Agglomerated particles |
| | | | observed, and clearing |
| | | | at bottom of the cylinder. |
| 100 mesh | 25 | min. | Suspension still homogeneous |
| Microencapsulated | 50 | min. | Suspension still homogenous |
| Linezolid (U.S. | | | except for some clearing at |
| standard 80-100 | | | bottom of the cylinder |
| mesh cut) | | | |
| | 24 | hrs. | Particles still pretty well |
| | | | suspended; very minimal |
| | | | clearing at bottom of the cylinder. |

It appears the smaller microencapsulated linezolid particles remained suspended in the suspension vehicle tested above; whereas, the largest sieve cut of particles tended to agglomerate and float. In the next Example, below, variations on the blend composition were tested to see whether it would be possible to produce and maintain a substantially homogeneous suspension of 80/100 mesh cut or larger microencapsulated linezolid particles for up to 24 hours after shaking.

### Example 4

### Effect of Particle Density on Homogeneity of Suspensions

The density of two different lots of non-sieved microencapsulated linezolid particles, and two different sieve cuts of microencapsulated particles was tested, using a Micrometricus AccuPyc 1330 apparatus. Table 4, below, summarizes the results of the density study.

TABLE 4

| | Microencapsulated | Weight of | Density of |
|---|---|---|---|
| | Linezolid | Sample | Sample |
| Sample | Particles Tested | (g) | (g/cm³) |
| 1 | Lot 1 mixture, 60 mesh cut | 2.73 | 1.371 |
| | (U.S. standard 30/60 mesh cut) | | |
| 2 | Lot 1 mixture, 100 mesh cut | 3.27 | 1.367 |
| | (U.S. standard 80/100 mesh cut) | | |
| 3 | Lot 1 mixture, not sieved | 2.46 | 1.372 |
| 4 | Lot 2 mixture, not sieved | 2.53 | 1.369 |

As one can see from Table 4, there was no significant difference between the density results obtained from each of the four different sets of particles tested. All were found to have a resultant density of 1.37 g/cm³, each of which was higher than the density of the suspension vehicle (1.16 g/cm³).

Since the density of the four samples of microencapsulated linezolid particles is higher that that of the suspension vehicle, one would expect the particles to sink in the vehicle. However, when each of the samples of particles described above was combined with the suspension vehicle and mixed in the presence of air, the particles did not sin. In fact, a substantially homogeneous suspension was formed.

### Example 5

### Identification of Dry Formulations that Produce Homogeneous Suspensions of Microencapsulated Linezolid

Three dry formulations of microencapsulated linezolid, with varying weight percent amounts of xanthan gum (Formulae B, C, and D) were produced as described in Example 2, above, using the component amounts composition shown in Table 5, below. Formula A, in Table 5, was produced using the same suspension vehicle used in all four formulations tested in Example 2, as described above. The same, non-sieved, mixture of microencapsulated linezolid particles was included in each of the four formulations tested in this study.

TABLE 5

| Formula A | | Formula B | | Formula C | | Formula D | | |
|---|---|---|---|---|---|---|---|---|
| mg/ | | mg/ | | mg/ | | mg/ | | |
| 5 ml | % by | 5 ml | % by | 5 ml | % by | 5 ml | % by | |
| Dose | Weight | Dose | Weight | Dose | Weight | Dose | Weight | Component |
| 218 | 10.9 | 218 | 10.9 | 218 | 10.9 | 218 | 10.9 | Linezolid |
| | | | | | | | | Microcapsules |
| 15.0 | 0.8 | 25.0 | 1.3 | 30.0 | 1.5 | 35.0 | 1.8 | Xanthan Gum |
| 50.0 | 2.5 | 40.0 | 2.0 | 35.0 | 1.8 | 30.0 | 1.5 | Microcrystalline |
| | | | | | | | | Cellulose and Sodium |
| | | | | | | | | Carboxymethylcellulose |
| | | | | | | | | (Avicel ® RC-591) |
| 935 | 46.8 | 935 | 46.8 | 935 | 46.8 | 935 | 46.8 | Sucrose |
| 500 | 25.0 | 500 | 25.0 | 500 | 25.0 | 500 | 25.0 | Mannitol |
| 15 | 0.8 | 15 | 0.8 | 15 | 0.8 | 15 | 0.8 | Sodium Citrate |
| 9.1 | 0.5 | 9.1 | 0.5 | 9.1 | 0.5 | 9.1 | 0.5 | Citric Acid |
| 10 | 0.5 | 10 | 0.5 | 10 | 0.5 | 10 | 0.5 | Sodium Benzoate |
| 13.5 | 0.7 | 13.5 | 0.7 | 13.5 | 0.7 | 13.5 | 0.7 | Sodium Chloride |
| 90 | 4.5 | 90 | 4.5 | 90 | 4.5 | 90 | 4.5 | Artificial Sweeteners |
| 94.5 | 4.8 | 94.5 | 4.8 | 94.5 | 4.8 | 94.5 | 4.8 | Natural & Artificial |
| | | | | | | | | Flavors |
| 2000 | (100) | 2000 | (100) | 2000 | (100) | 2000 | (100) | Total Powder Weight |
| | | | | | | | | per 5 ml Dose |

66 g of each Formulation was placed in a container, and suspended in 120 ml of water, by mixing in the presence of air. 25 to 30 ml of each resulting suspension was transferred to 50 ml graduated cylinders and visually examined for air bubble distribution. After 4 days undisturbed, a sample of each suspension was withdrawn from the bottom of each graduated cylinder and examine microscopically. The following observations were made:

For Formulation A, large particles were observed floating to the top of the graduated cylinder within 5 minutes of the formula being placed in the 50 ml cylinder.

For Formulation B, the following observations were made. Not many air bubbles were present initially, and those that were present were concentrated in the top 10 ml of the cylinder. Air bubbles migrated at a rate of about 1 mm/minute. After 4 days undisturbed, no air bubbles remained in the cylinder. Microscopic examination of suspension from the bottom of the original bottle after 4 days undisturbed revealed no significant amount of air, and no aggregation of microcapsules.

For Formulation C, the following observations were made. Many air bubbles were present initially, uniformly distributed throughout the graduated cylinder. Migration was not visually detectable over the first 15 minute time frame. After 4 days of standing undisturbed, many bubbles were still present, but had migrated upward somewhat so that the bottom 5 ml of the cylinder was devoid of visible air bubbles. Microscopic examination of the suspension drawn from the bottom of the original bottle of Formulation C after 4 days undisturbed revealed the presence of air bubbles, and no aggregation of microcapsules.

Observations of Formulation D were identical to those made with regard to Formulation C, summarized above. However, Formulation D was found to be so viscous that with lot to lot variability of xanthan gum, the formulation could potentially even gel.

Use tests of all three test formulations described above (i.e., Formulations B through D) showed a very little sample to sample variance compared to control Formulation A, regardless of which of at least two different mixing techniques was used to mix each formulation prior to drawing a sample for testing. However, Formulation C performed the best of the four formulations tested, in producing a substantially homogeneous, not overly viscous, suspension of the microencapsulated linezolid particles that appeared to maintain its homogeneity over time, after combination with water and shaking in the presence of air.

### EXAMPLE 6

### Effect of Viscosity on Maintaining Suspension Homogeneity

Three different samples of Formula C were prepared as described in Example 5, above, using two different lots of microencapsulated linezolid particles that had not been sieve cut, and different lots of xanthan gum and Avicel® RC-591. The viscosity of each resulting suspension was found to vary from one sample to another, as follows:

Sample 1 was found to have a viscosity of 2500 to 2800 cps.

Sample 2 was found to have a viscosity of 3540 cps.

Sample 3 was found to have a viscosity of 2800 to 3000 cps.

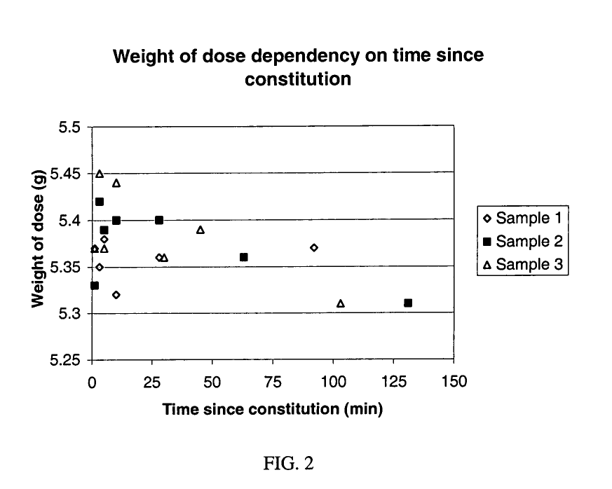

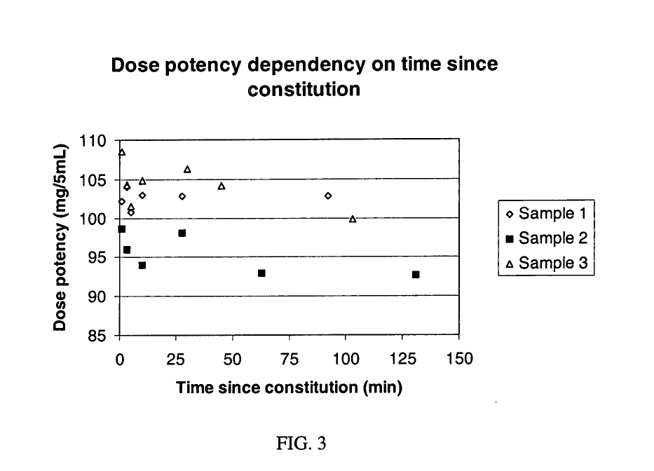

Each suspension sample prepared as described above was allowed to stand at room temperature. 5 ml. aliquots of each sample were taken at various time points, and tested for weight and dose potency of linezolid in each aliquot. Duplicate samples were taken at each final time tested. The results of this study show that weight of dose dependency and dose potency dependency on time since constitution was minimal in all three samples tested. Results of this assay are summarized in Table 6, below. Results of the weight of dose dependency study are illustrated in FIG. 2. Results of the dose potency study are illustrated in FIG. 3.

TABLE 6

| Sample | Elapsed | Weight of | Dose |
|---|---|---|---|
| Number | Time (min.) | Dose (g.) | Potency |
| 1 | 1 | 5.37 | 102.2 |
| | 3 | 5.35 | 104.0 |
| | 5 | 5.38 | 100.7 |
| | 10 | 5.32 | 103.0 |
| | 28 | 5.36 | 102.8 |
| | 92 | 5.37 | 102.8 |
| | 221 | 5.40 | 101.4 |
| | 1433 | 5.42 | 99.5 |
| | 1433 | 5.33 | 98.5 |
| Mean | | 5.37 | 101.4 |
| Std. Dev. | | 0.030 | 3.58 |
| 2 | 1 | 5.33 | 98.6 |
| | 3 | 5.42 | 96.0 |
| | 5 | 5.39 | 63.2 |
| | 10 | 5.40 | 94.0 |
| | 28 | 5.40 | 98.1 |
| | 63 | 5.36 | 92.9 |
| | 131 | 5.31 | 92.7 |
| | 1154 | 5.31 | 93.4 |
| | 1154 | 5.35 | 89.6 |
| Mean | | 5.63 | 94.3 |
| Std. Dev. | | 0.041 | 2.83 |
| 3 | 1 | 5.37 | 108.5 |
| | 3 | 5.45 | 104.3 |
| | 5 | 5.37 | 101.6 |
| | 10 | 5.44 | 104.8 |
| | 30 | 5.36 | 106.3 |
| | 45 | 5.39 | 104.2 |
| | 103 | 5.31 | 99.8 |
| | 1442 | 5.46 | 106.0 |
| | 1442 | 5.29 | 96.9 |
| Mean | | 5.38 | 103.6 |
| Std. Dev. | | 0.060 | 3.58 |

As one can see from the results shown in Table 6 and plotted in FIGS. 2 and 3, the dose weight and potency per each 5 ml aliquot taken from each of the three sample suspensions varied very little over the time period tested after initial mixing in the presence of air. The standard deviation (“Std. Dev.”) of both the weight of dose and dose potency results in extremely low and comparable for all three samples tested. The microencapsulated linezolid particle mixtures tested in this study remained suspended in various preparations of the suspension vehicle tested in this study, preparations produced using different lots of xanthan gum, Avicel® RC-591, and microencapsulated particles, even though the viscosity of each resulting suspension varied from sample to sample.

We claim:

1. A dry formulation, comprising:
at least two doses of coated drug particles having an average particle size of about 50 μm to about 600 μm, each coated drug particle comprising a core comprising a drug, and a hydrophobic polymer film coating at least a portion of the core; and
a viscosity enhancing substance in an amount effective to maintain the at least two doses of coated drug in a substantially homogeneous suspension for at least 24 hours at about 20° C. to about 30° C., after combination with about 2 ml to about 60 ml of an aqueous liquid per dose of the coated drug and mixing in the presence of air.

2. The dry formulation of claim 1, wherein the hydrophobic polymer film is selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

3. The dry formulation of claim 1, wherein the hydrophobic polymer film comprises methacrylic acid or a polymethacrylic acid copolymer.

4. The dry formulation of claim 1, wherein the hydrophobic polymer film further comprises a plasticizer.

5. The dry formulation of claim 1, wherein the drug is an oxazolidinone antibiotic.

6. The dry formulation of claim 5, wherein the oxazolidinone antibiotic is linezolid.

7. The dry formulation of claim 1, wherein the viscosity enhancing substance is selected from the group consisting of an alginate, carageenin, agar-agar, tragacanth gum, xanthan gum, guar gum, caroba gum, karaya gum, modified corn starch, carboxymethyl cellulose, and crystalline cellulose alone or in combination with other hydrocolloids.

8. The dry formulation of claim 1, wherein the viscosity enhancing substance is a mixture of xanthan gum, microcrystalline cellulose, and sodium carboxymethylcellulose.

9. The dry formulation of claim 8, wherein the weight ratio of the xanthan gum to the microcrystalline cellulose and the carboxymethylcellulose in the mixture is about 1:2 to about 1:0.3.

10. The dry formulation of claim 1, wherein the viscosity enhancing substance is present in an amount such that the suspension has a viscosity of at least about 1500 cps.

11. The dry formulation of claim 10, wherein the viscosity enhancing substance is present in an amount such that the suspension has a viscosity of at least about 1500 cps to about 4000 cps.

12. The dry formulation of claim 1, wherein the coated drug particles have an average particle size of about 100 μm to about 600 μm.

13. The dry formulation of claim 1, further comprising at least one taste-masking substance.

14. The dry formulation of claim 13, wherein the at least one taste-masking substance is a sugar.

15. The dry formulation of claim 14, wherein the sugar is sucrose.

16. The dry formulation of claim 13, wherein the taste-masking substance is an artificial sweetener.

17. The dry formulation of claim 13, wherein the at least one taste-masking substance is a flavoring agent.

18. The dry formulation of claim 1, wherein the aqueous liquid is water.

19. The dry formulation of claim 1, wherein the coated drug particles are suspended in the aqueous liquid within five (5) minutes of combination of the dry formulation with the aqueous liquid.

20. The dry formulation of claim 1, wherein the dry formulation forms a suspension with a homogeneous dispersion of air bubbles and solid particles that do not interact with each other after the mixing in the presence of the air.

21. A dry formulation, comprising:
at least two doses of coated drug particles having an average particle size of about 50 μm to about 600 μm, each coated drug particle comprising a core comprising a drug, and a hydrophobic polymer film coating at least a portion of the core;
xanthan gum; and
a combination of microcrystalline cellulose and sodium carboxymethylcellulose;
wherein the weight ratio of the xanthan gum to the combination of microcrystalline cellulose and sodium carboxymethylcellulose is about 1:2 to about 1:0.2, and wherein a suspension with a viscosity of at least about 1500 cps is formed after combination of the dry formulation with about 2 ml to about 60 ml of an aqueous liquid per dose of the coated drug particles.

22. The dry formulation of claim 21, wherein the suspension formed after combination of the dry formulation with the aqueous liquid has a viscosity of about 1500 cps to about 4000 cps.

23. The dry formulation of claim 21, wherein the hydrophobic polymer is selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

24. The dry formulation of claim 21, wherein the hydrophobic polymer is methacrylic acid or a polymethacrylic acid copolymer.

25. The dry formulation of claim 21, the hydrophobic polymer film further comprising a plasticizer.

26. The dry formulation of claim 21, wherein the drug is an oxazolidinone antibiotic.

27. The dry formulation of claim 21, wherein the oxazolidinone antibiotic is linezolid.

28. The dry formulation of claim 21, wherein the combination of microcrystalline cellulose and sodium carboxymethylcellulose is Avicel® RC-591.

29. The dry formulation of claim 21, further comprising at least one taste-masking substance.

30. The dry formulation of claim 30, wherein the at least one taste-masking substance is a sugar.

31. The dry formulation of claim 30, wherein the sugar is sucrose.

32. The dry formulation of claim 29, wherein the taste-masking substance is an artificial sweetener.

33. The dry formulation of claim 29, wherein the at least one taste-masking substance is a flavoring agent.

34. The dry formulation of claim 21, wherein the dry formulation forms a suspension with a homogeneous dispersion of air bubbles and solid particles that do not interact with each other after combination with the aqueous solution and mixing in the presence of air.

35. A method of producing a multi-dose suspension, comprising the steps of:
a) providing a dry formulation comprising:
at least two doses of coated drug particles having an average particle size of about 50 μm to about 600 μm, each coated drug particle comprising a core comprising a drug, and
a hydrophobic polymer film coating at least a portion of the core, xanthan gum, and a combination of microcrystalline cellulose and sodium carboxymethylcellulose,
wherein the weight ratio of the xanthan gum to the combination of microcrystalline cellulose and sodium carboxymethylcellulose is about 1:2 to about 1:0.2; and
b) combining the dry formulation with an aqueous solution and agitating the same until a suspension is formed, having a viscosity of at least about 1500 cps.

36. The method of claim 35, wherein the hydrophobic polymer is selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

37. The method of claim 35, wherein the hydrophobic polymer is methacrylic acid or a polymethacrylic acid copolymer.

38. The method of claim 35, wherein the drug is an oxazolidinone antibiotic.

39. The method of claim 38, wherein the oxazolidinone antibiotic is linezolid.

40. The method of claim 35, wherein the coated drug particles have an average particle size of about 100 μm to about 600 μm.

41. The method of claim 35, wherein the viscosity of the suspension formed in step (b) is at least about 1000 cps.

42. The method of claim 35, wherein the dry formulation provided in step (a) further comprises at least one taste-masking substance.

43. The method of claim 42, wherein the at least one taste-masking substance is a sugar.

44. The method of claim 43, wherein the sugar is sucrose.

45. The method of claim 35, wherein the aqueous solution is water.

46. The method of claim 35, wherein the suspension is substantially homogeneous.

47. The method of claim 46, wherein the suspension comprising a homogeneous dispersion of air bubbles and solid particles that do not interact with each other after mixing in the presence of the air.

48. A method of treating or preventing a gram-positive bacterial infection, comprising orally administering at least two doses of a multi-dose suspension to a subject, the multi-dose suspension comprising:
at least two doses of coated oxazolidinone antibiotic drug particles suspended in about 2 ml to about 60 ml of an aqueous liquid per dose of the coated drug, the coated drug particles having an average particle size of about 50 μm to about 600 μm, each coated drug particle comprising a core comprising an oxazolidinone antibacterial drug, and a hydrophobic polymer film coating at least a portion of the core; and
a viscosity enhancing substance in an amount effective to maintain the at least two doses of coated oxazolidinone antibiotic drug particles in a substantially homogeneous suspension in the aqueous solution for at least 24 hours at about 20° C. to about 30° C., after combination with and mixing in the presence of air.

49. The method of claim 48, wherein the oxazolidinone antibiotic drug is linezolid.

50. The method of claim 48, wherein the multi-dose suspension is orally administered to treat or prevent infection of the subject by at least one bacteria from a genus selected from the group consisting of: Staphylococcus, Streptococcus, Enterococcus, Bacillus, Corynebacterium, Chlamydia and Neisseria.
